(19) United States
(12) Patent Application Publication
(10) Pub. No.: US 2013/0137966 A1
(43) Pub. Date: May 30, 2013

(54) TISSUE EXTRACTION SYSTEM AND THREE-DIMENSIONAL DISPLAY METHOD OF THE SAME

(71) Applicants: Ryuichi Nakahara, Okayama (JP); Keiichiro Nishida, Okayama (JP); Toshifumi Ozaki, Okayama (JP)

(72) Inventors: Ryuichi Nakahara, Okayama (JP); Keiichiro Nishida, Okayama (JP); Toshifumi Ozaki, Okayama (JP)

(73) Assignees: Ryuichi NAKAHARA, Okayama-shi, Okayama (JP); AZE LTD., Tokyo (JP); Toshifumi OZAKI, Okayama-shi, Okayama (JP); Keiichiro NISHIDA, Okayama-shi, Okayama (JP)

(21) Appl. No.: 13/812,078

(22) PCT Filed: Jul. 29, 2011
(86) PCT No.: PCT/JP2011/067526

§ 371 Date: Jan. 24, 2013

(30) Foreign Application Priority Data
Jul. 30, 2010 (JP) 2010-172482

(51) Int. Cl. A61B 5/055

(52) U.S. Cl. CPC A61B 5/055
USPC 600/411

(57) ABSTRACT

A tissue extraction system for extracting tissue from a subject by using an image, wherein the tissue extraction system comprises: an imaging unit for arranging coordinate axes to capture a plurality of sequences; a conversion unit for converting the plurality of sequences captured by the imaging unit into histogram space in which the sequences are described in terms of the coordinate axes; and an extraction unit for extracting the tissue of the subject from the distribution of the histogram converted by the conversion unit.

### TECHNICAL FIELD

The present invention relates to a tissue extraction system for extracting tissue of a subject by using images, and a three-dimensional display method of the same.

### TECHNICAL BACKGROUND

Magnetic resonance imaging (MRI) systems typically capture various MRI images having different levels of tissue contrast in the manner of T1 weighted images or T2 weighted images (to be referred to as an “MRI sequence” or “sequence”) as sets of images (see Patent Document 1). Since MRI images were initially used for diagnosis by developing on black and white film, diagnoses are still made based on black and white images even at present when diagnoses are made with a display.

Since the values of MRI signals vary for each test, tissue was unable to be diagnosed with these signal values alone. Therefore, MRI signals are used for diagnosis by referring to as “High” in the case signal values are higher than those of muscle, as “Low” in the case they are lower, or as “Iso” in the case they are equal. More specifically, areas on MRI images that are whiter than muscle are referred to as “High”, areas that are blacker as “Low”, and areas of about the same coloring as “Iso”. As a result, areas that are “High” in T1 weighted images and “High” in T2 weighted images can be presumed to indicate fat components, while areas that are “Low” in T1 weighted images and “High” in T2 weighted images can be presumed to indicate water components.

Various sequences have been developed that have specific tissue contrast, such as T2* (star) weighted images, diffusion weighted images, or T2 fat suppression, in order to enhance diagnostic performance, and are continuing to increase at present. At present, as shown in FIG. 11, diagnoses are made by capturing images of a plurality of sequences according to the objective, and then visually comparing each sequence with black and white images.

### PRIOR ARTS LIST

### Patent Document

Patent Document 1: Japanese Laid-Open Patent Publication No. H7-505805(A) based on PCT International Application

### SUMMARY OF THE INVENTION

### Problems to be Solved by the Invention

As has been described above, tissue is normally identified by visually comparing a plurality of sequences. However, according to this type of method, it is necessary to look at individual sequences of the plurality of sequences and superimpose them mentally, thereby resulting in cases in which it is difficult to identify tissue immediately.

With the foregoing in view, an object of the present invention is to provide a tissue extraction system capable of efficiently extracting tissue using images and a three-dimensional display method of the same.

### Means to solve the Problems

The present invention is a tissue extraction system for extracting tissue of a subject using images, including: an imaging unit for capturing a plurality of sequences by arranging coordinate axes; a conversion unit for converting the plurality of sequences captured by the imaging unit into histogram space in which the sequences are described in terms of the coordinate axes; and an extraction unit for extracting the tissue of the subject from the distribution of the histogram generated by the conversion unit.

### Advantageous Effects of the Invention

According to the present invention, since tissue isolation efficiency is enhanced as a result of a plurality of sequences being converted to histogram space, a tissue extraction system and three-dimensional display method thereof can be provided that are capable of efficiently extracting tissue.

### BRIEF DESCRIPTION OF THE DRAWINGS

FIG. 1 is a block diagram of a tissue extraction system in an embodiment of the present invention;

FIG. 2 is a flow chart showing processing of histogram space in an embodiment of the present invention;

FIG. 3(A) is an explanatory drawing showing development in the histogram space in an embodiment of the present invention prior to that development, while FIG. 3(B) is an explanatory drawing showing development to histogram space in an embodiment of the present invention after that development;

FIG. 4(A) is a drawing for explaining a technique for analyzing islands (water) in an embodiment of the present invention, FIG. 4(B) is a drawing for explaining a technique for analyzing islands (fat) in an embodiment of the present invention, FIG. 4(C) is a drawing for explaining a technique for analyzing islands (bone marrow) in an embodiment of the present invention, FIG. 4(D) is a drawing for explaining a technique for analyzing islands (muscle/cartilage) in an embodiment of the present invention, and FIG. 4(E) is a drawing for explaining a technique for analyzing islands (air/bone/ligament) in an embodiment of the present invention;

FIG. 5 is a drawing for explaining a technique for analyzing islands in an embodiment of the present invention;

FIG. 6 is a drawing showing center points of islands in an embodiment of the present invention;

FIG. 7(A) is a drawing showing an example of a screen display on a display unit in an embodiment of the present invention, while FIG. 7(B) is a drawing showing another example of a screen display on a display unit in an embodiment of the present invention;

FIG. 8 is a drawing showing the relationship between combinations of cone cells and color;

FIG. 9 is a drawing for explaining tissue classification in an embodiment of the present invention;

FIG. 10 is a drawing for explaining WL and WW in an embodiment of the present invention; and

FIG. 11 is a drawing for explaining interpretation of MRI images.

### DESCRIPTION OF THE EMBODIMENTS

The following provides an explanation of an embodiment of the present invention with reference to the drawings.

(1) Configuration of Tissue Extraction System

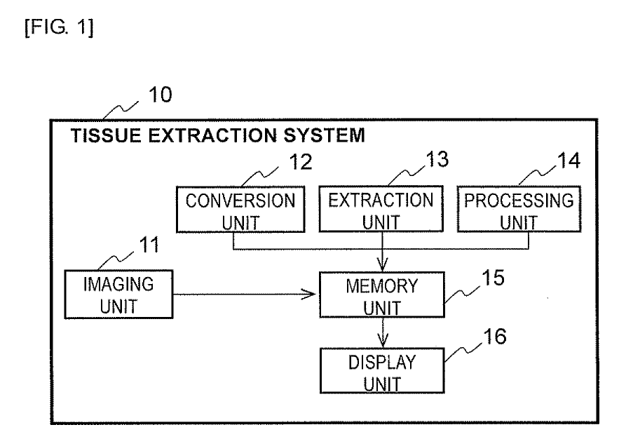

FIG. 1 is a block diagram of a tissue extraction system 10 in an embodiment of the present invention. This tissue extraction system 10 is a system for extracting tissue of a subject using MRI images, and functionally, is provided with an imaging unit 11, a conversion unit 12, an extraction unit 13, a processing unit 14, a memory unit 15 and a display unit 16. Naturally, although other processing units such as an input unit for inputting information or a communication unit for communicating with other systems may also be provided, they are not shown here.

The imaging unit 11 is an MRI unit and the like for capturing a plurality of MRI sequences by arranging coordinate axes. The conversion unit 12 converts the plurality of MRI sequences captured by the imaging unit 11 into histogram space in which the MRI sequences are described in terms of the coordinate axes. The extraction unit 13 extracts tissue of the subject from the distribution of the histogram converted by the conversion unit 12. The processing unit 14 carries out various types of processing using results generated by the extraction unit 13. The memory unit 15 stores various data such as processing results of each processing unit. The display unit 16 is a liquid crystal display and the like that displays data stored in the memory unit 15. The conversion unit 12, the extraction unit 13, the processing unit 14, the memory unit 15 and the display unit 16 can be connected to an image processing workstation. An image processing workstation is a dedicated device for carrying out sophisticated image processing, the details of which will be subsequently described.

(2) Characteristics of CT Images and Image Processing

Prior to providing a detailed explanation of the configuration of the tissue extraction system 10, an overview is provided of the functions of computed tomography (CT) along with an explanation of those circumstances that have led to the growing importance of image processing. X-ray photographs constitute a two-dimensional system in which a subject is irradiated with radiation, and images are captured on a film that is sensitive to radiation placed behind the subject. CT constitutes a system for generating cross-sectional images by capturing images while rotating a radiation source and a receiver and then calculating the resulting data. Since data had a slice thickness of 3 mm to 5 mm until the 1990s, the number of images for a single test rarely exceeded 100, and diagnoses were made by developing the images on film.

Subsequently, helical CT was developed, slice thickness was reduced to 0.5 mm to 1 mm, and imaging time became shorter by reducing the exposure dose by employing multiple rows of detectors. For example, imaging from the head to the feet was able to be completed in just over 10 seconds, and the number of images came to exceed 1000. As a result, even if a subject was unconscious from a traffic injury and the like and it was not possible to determine the location of the injury, it became possible to make a diagnosis of the head, chest, abdomen and pelvis, etc. all at once, resulting in an improvement of survival rate. However, in the case of more than 1000 images, since developing those images onto film created problems in terms of cost, medical displays developed for the purpose of displaying medical images became popular.

On the other hand, in the case of diagnosing a compound fracture, since it was difficult to arrange CT images consisting of a collection of two-dimensional images and mentally construct the three-dimensional structure of bone, a demand arose for the production of three-dimensional images based on CT images. Although only three-dimensional images having low resolution were able to be generated when slice thickness was relatively thick, as a result of slice thickness becoming progressively thinner, it became possible to generate extremely concise three-dimensional images.

CT image signals are referred to as Hounsfield values (HU values) and, differing from MRI, since they are standardized so that water has a value of 0 and air has a value of −1000, the signal distributions of muscle and bone are known in advance. If only sites are extracted that have a bone signal distribution (roughly 200 or more), then only areas of bone are obtained, and the three-dimensional structure of the bone can be generated automatically on the basis of that information. Similarly, since the signal distributions of internal organs or muscle are also known, it is possible to automatically generate three-dimensional structures of specific tissues. Consequently, three-dimensional representation becomes easier the greater the degree to which signal distribution of a certain tissue does not overlap that of other tissue. Since the signal distribution of blood vessels resembles that of muscle and other soft tissue, it is difficult to extract only blood vessels. Therefore, contrast medium that is safe for use in humans and exhibits high CT values can be used to increase the contrast of blood vessels by administering intravenously immediately prior to CT imaging. The signal value of contrast medium resembles the signal distribution of bone. Consequently, although it is difficult to extract only blood vessels from contrast CT images alone, only blood vessels can be extracted by calculating back using CT images obtained before administration of contrast medium and CT images obtained after administration of contrast medium. Image processing workstations are dedicated devices for carrying out such sophisticated image processing. Furthermore, testing using contrast medium is also possible with MRI.

(3) Standardization of Medical Image Standards and the Rise of the Image Processing Workstation Industry

Next, an explanation is provided to obtain an understanding of the current situation by providing an overview of the history of the clinical imaging market. In the past, medical imaging system manufacturers only manufactured systems based on their own standards, and as a result of each firm attempting to secure a share of the market, individual medical institutions were only able to use systems of the same manufacturer, thereby resulting in increased costs. Therefore, discussions were held between the American College of Radiology and the National Electrical Manufacturers Association that resulted in the establishment of the Digital imaging and Communications in Medicine (DICOM) standard. As a result, it became possible to exchange images between imaging systems of different manufacturers.

This had two effects on the market. The first was an increase in market size. Since conformity to the DICOM standard made it possible to market products in the U.S. and Europe as well as Asia, market size increased rapidly. The second effect was increased division of labor among manufacturers. Manufacturers divided their operations into specialized firms in the form of MRI, CT, image server, image processing workstations and medical display firms. These two elements combined to give rise to manufacturers of medical imaging-related products throughout the world and accelerated the pace of technical innovations. At present, large hospitals in Japan have introduced image processing workstations, and have divided their operations into departments responsible for CT and MRI imaging and departments responsible for three-dimensional and other image processing.

Revenues of image processing workstation firms are generated from support costs. Although these workstations are normally shipped with dedicated software installed in a high-performance PC, since enhancements to PC performance occur every few years, even though processing speed may be the fastest at the time of purchase, it soon becomes outdated. Consequently, numerous business models are employed that generate maintenance and inspection fees by enabling updates every few years. Manufacturers are enhancing the market value of their products by adding various imaging processing functions in order to distinguish them from other firms. Although these workstations were initially only provided with functions for generating three-dimensional representations of CT images, they have recently been equipped with MRI image processing functions as well. The present invention can be applied as an add-on to image processing software installed in such image processing workstations.

(4) Technique for Processing MRI Images in the Manner of CT Images

The following provides an explanation of standardization of MRI image signals using histogram space. The problem with MRI images is that their signals are relative signals that change for each test. Namely, since the signal values of CT images are standardized to a value of 0 for water and a value of −1000 for air, specific tissues can be extracted relying only on signal values. On the other hand, in the case of MRI, it is difficult to automatically extract specific tissues based on signal values alone, and automatic extraction requires knowledge of the signal distribution for each tissue.

Therefore, in the tissue extraction system 10 in an embodiment of the present invention, a technique is employed for extracting tissue from a histogram (frequency distribution) of MRI image signals. Namely, instead of standardizing on the basis of signal values obtained by capturing a specific substance as in the manner of HU values, the object is to isolate tissue based on signal distribution.

When a histogram of a single MRI sequence is generated, distributions in which a series of peaks can be obtained. However, since the distributions overlap over a wide range, it is difficult to isolate each tissue distribution based on the distribution of a histogram of a single sequence. This is caused by each of the tissue signals having a wide distribution and mutual signal distributions overlapping. Consequently, it has been considered to be difficult to isolate tissue from this histogram.

However, if a plurality of sequences is captured by arranging coordinate axes, tissue can be isolated from that histogram. “Capturing by arranging coordinate axes” refers to capturing images while fixing the imaging conditions of the subject (including imaging range and imaging magnification factor). If the signal values of each sequence are defined as (x,y,z)=Sn (where, S represents a signal value determined by specified coordinates, and n represents the sequence number), then integrating each sequence results in (x,y,z)=(S1, S2, . . . , Sn). Therefore, sequences are converted to histogram space H (S1, S2, . . . , Sn) in which the sequences are described in terms of the coordinate axes. As a result, tissues for which signal distributions overlap are isolated in the histogram space for each sequence. Namely, two groups that are unable to be distinguished with a single variable can be converted using multiple variables. In the present invention, the use of this type of multivariate analysis makes it possible to enhance tissue isolation efficiency by converting a plurality of MRI sequences to histogram space.

If a site at which images have been captured is known, since the type of tissue present at that site is known, which distribution corresponds to a specific tissue can be identified from the signal pattern in the histogram space. If a collection of signals in the histogram space is referred to as an “island”, then which island corresponds to which tissue can be determined. Although the precise location of an island changes depending on subtle changes in imaging sequence settings, the distribution pattern does not change. For example, when a T1 weighted image, T2 weighted image and T2* (star) weighted image of the knee joint are captured and converted to histogram space, islands are formed consisting of water, bone marrow, fat, muscle and the like. In the case muscle has degenerated with age, although the distance between islands changes, the sequential relationship of island locations does not change that much. Signal values can be standardized and tissue can be extracted by using this property.

(5) Processing of Histogram Space

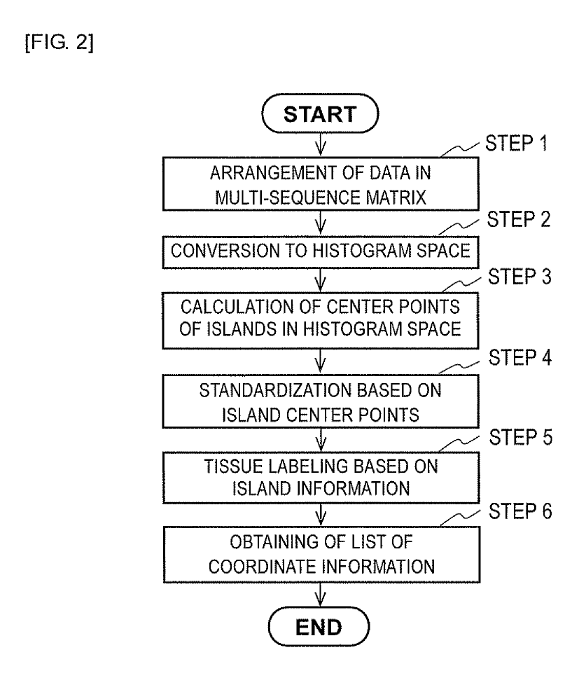

FIG. 2 is a flow chart showing processing of histogram space in an embodiment of the present invention. To label tissue with respect to coordinates is essentially important to obtain a mask The following provides a detailed explanation of each type of processing.

(5-1) Development in the Histogram Space

Signal values of each sequence are defined as (x,y,z,)=Sn. Since the minimum and maximum values of Sn change according to the sequence, type of MRI and settings, ultimately Sn was considered to be converted to a standardized rSn (stage 5-2 indicated below).

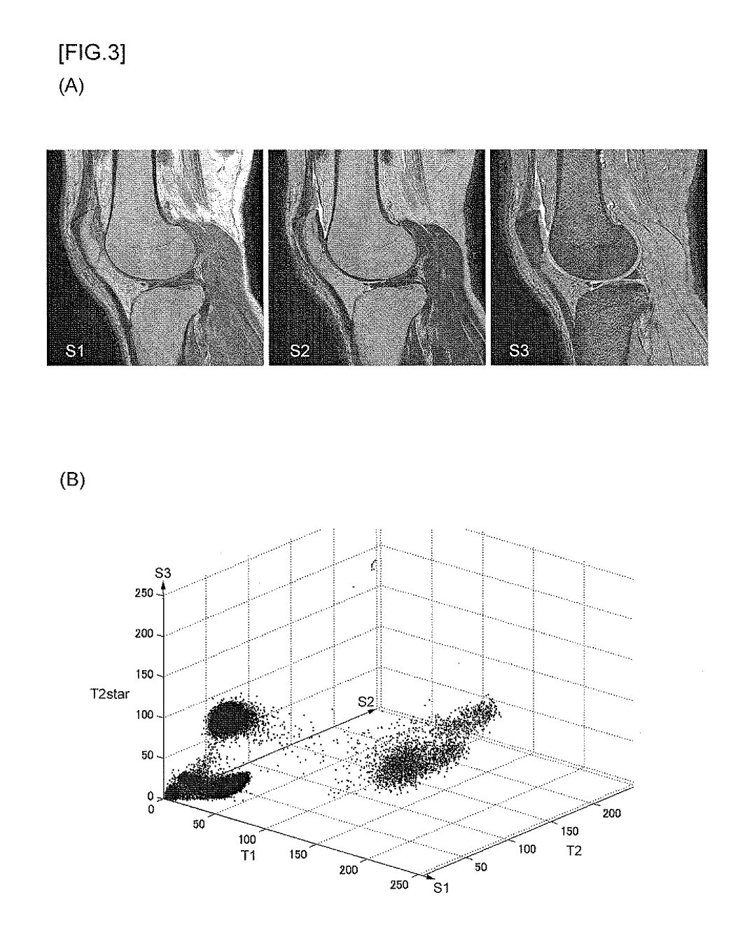

Therefore, as shown in FIG. 3(A), the conversion unit 12 integrates a plurality of sequences and arranges the data in a multi-sequence matrix S (x,y,z)=(S1, S2, . . . , Sn) (FIG. 2, Step 1). Next, as shown in FIG. 3(B), the sequences are developed by calculating frequency distributions of the histogram space H (S1, S2, . . . , Sn)=m (FIG. 2, Step 2).

(5-2) Standardization of Island Analysis

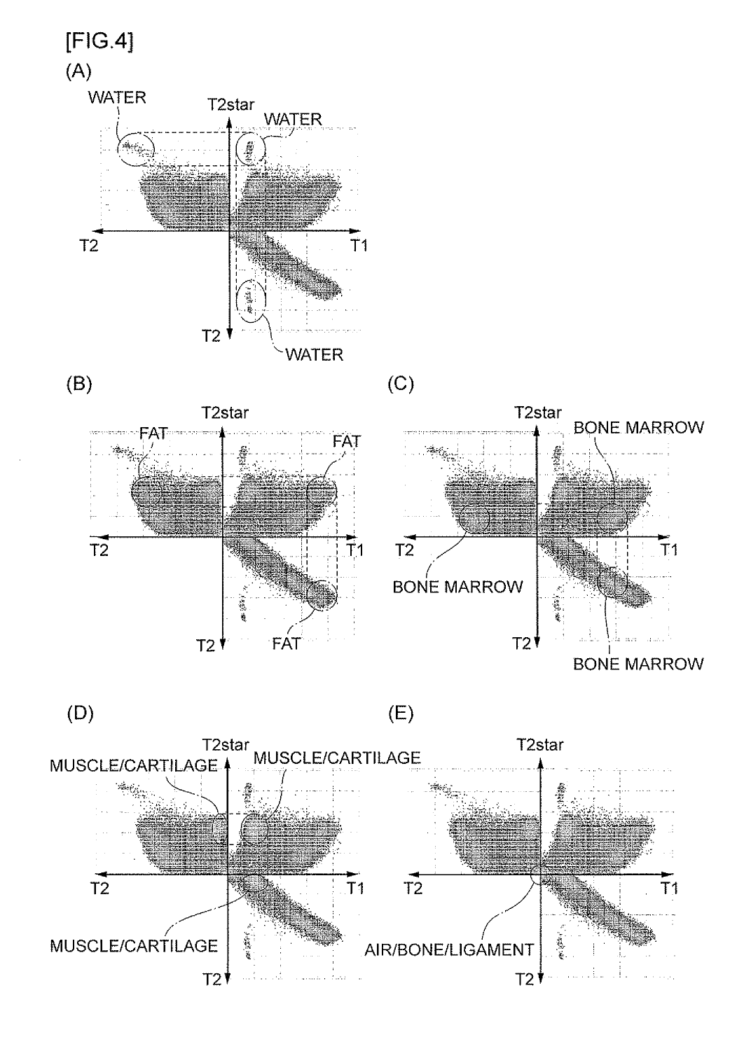

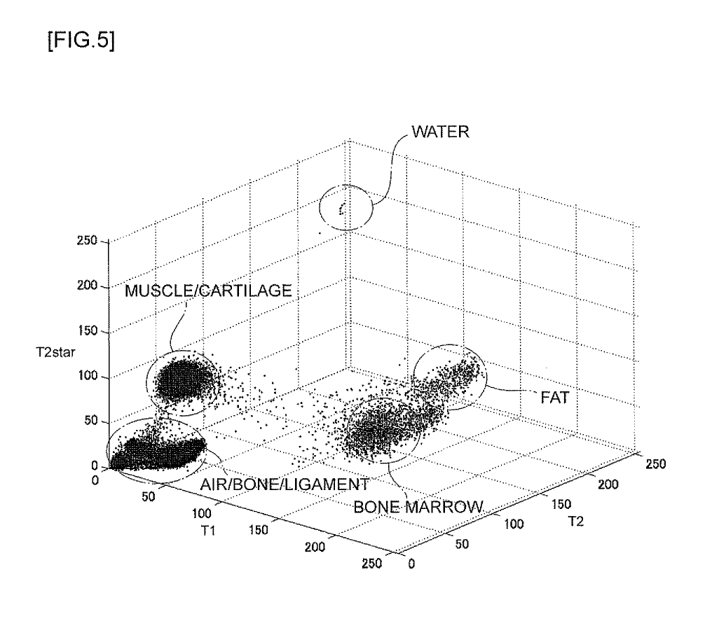

Since the histogram space consists of distributions having a large number of gaps, isolation becomes easier than in the case of a single sequence. Although there are differences between individuals, such as some individuals having a large amount of muscle or some individuals having a large amount of bone, signal distribution or island distribution in the histogram space does not change. However, since signal values are not yet standardized at this stage, the tissue name of an island is first identified based on the distribution status of the island. In looking at the example of a joint, in the case the sequences consist of a T1 weighted image, T2 weighted image and T2* weighted fat suppression image, then as shown in FIG. 4, water in FIG. 4A, fat in FIG. 4B, bone marrow in FIG. 4C, muscle/cartilage in FIG. 4D and air/bone/ligament in FIG. 4E are separated into respectively different islands. A three-dimensional representation of this histogram is shown in FIG. 5.

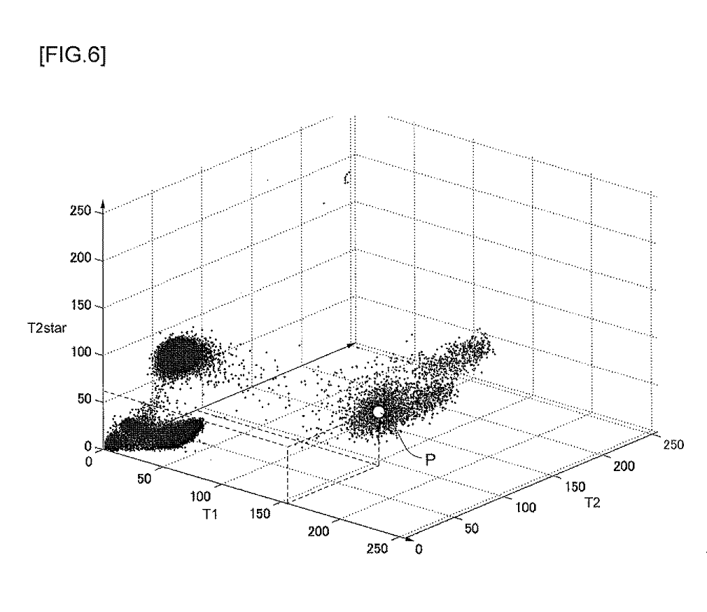

Therefore, as shown in FIG. 6, the extraction unit 13 calculates the center point P of an island in the histogram space, and carries out standardization based on that center point P (FIG. 2, Steps 3 and 4). Although error occurs to a certain degree in each test, since the center points P do not change that much, they are useful for standardization.

More specifically, standardization is carried out using island center data for each tissue generated based on a healthy individual. For example, when a T1 weighted image, T2 weighted image and T2* weighted fat suppression image were captured by 1.5 T MRI using a knee coil, the center of the fat signal of the knee is assumed to be at H (200, 200, 100) in the histogram space. In the case island center data of the fat signal of the knee in that MRI was rH (150, 200, 60), then the conversion variable A becomes A=(0.75, 1.0, 0.6) from “A*H=rH”. Reproducibility increases if a tissue having a large signal value at the island center is selected. Although this method is based on linear transformation, non-linear transformation using similarities of distribution patterns is effective for further enhancing standardization accuracy.

(5-3) Tissue Labeling

Next, the extraction unit 13 carries out labeling for correlating histogram space coordinates with tissue (FIG. 2, Step 5). It is difficult to exclusively specify whether muscle or bone and the like are present in a certain space. Regardless of the manner in which the accuracy of an imaging test is improved, since voxel (volume pixel) size cannot be decreased to the level of cells, various tissues end up being present in a certain voxel. Therefore, a method was considered in which existence probabilities of each tissue are specified for each voxel.

Namely, an existence probability is determined for each tissue based on the distance from the center of an island in the histogram space. The probability of the vicinity of the center of an island being tissue is assumed to be 100%. Since information on the distribution of surrounding signals is required to specify probability in the case of being at a distance from an island center, existence probability cannot simply be determined based on distance from an island center. This is because, in the case two tissues are in close proximity, the existence probabilities of those tissues decreased rapidly moving away from the center. Existence probability patterns change depending on which type of tissue is present at the imaging site.

Since tissue classification in the histogram space changes slightly for each imaging site in this manner, a probability distribution library is created in the case of having determined and standardized island centers of tissues for each site. As a result, tissue labeling can be carried out automatically using the probability distribution library.

A probability distribution library is not required in the case of carrying out tissue labeling manually instead of automatically. For example, signal distribution in histogram space can be made to be displayed on the display unit 16, and a user can specify ranges corresponding to certain tissues in the histogram space while viewing the signal distribution. This method must be used at sites for which a probability distribution library is not available.

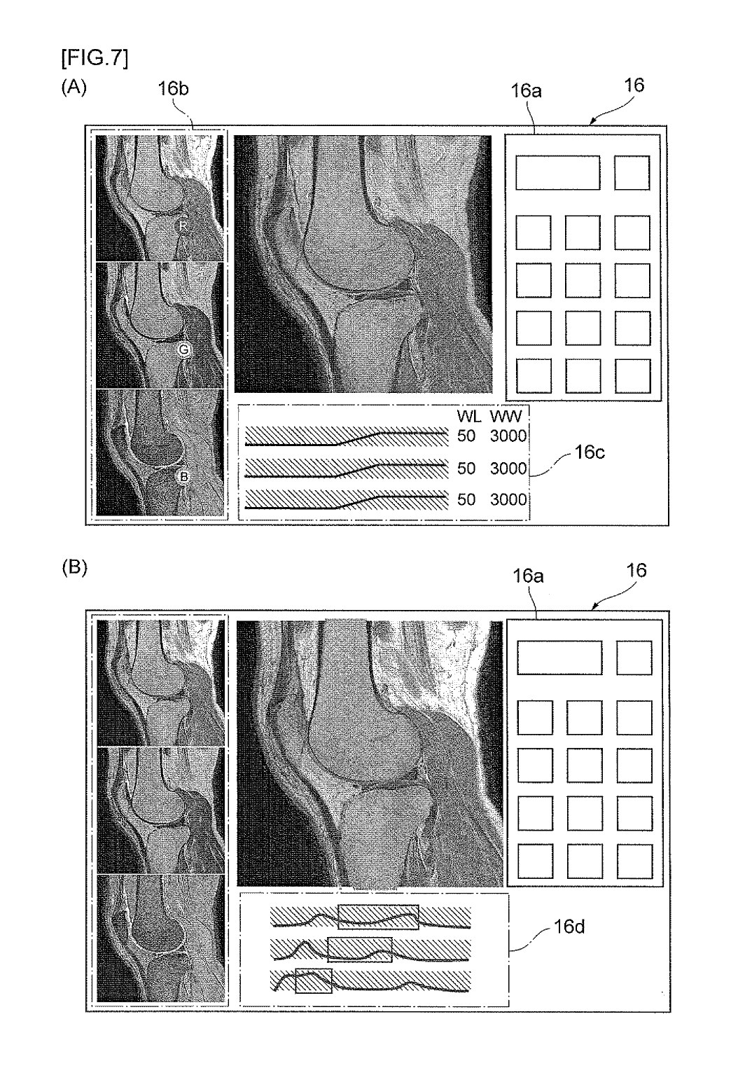

FIG. 7 is a drawing showing examples of screen displays on the display unit 16. The display unit 16 is able to display histogram space using a trihedral figure and the like. As shown in these drawings, a group of operating buttons is arranged in a display area 16a, and various operations can be instructed using this group of operating buttons. For example, color schemes for each sequence can be selected in a display area 16b, while the signal conversion level and gamma correction of each sequence can be set in a display area 16c (to be subsequently described). Moreover, the specific tissue distribution of histogram space can be specified by selecting and enclosing a specific signal range (rectangular area in the drawings) in a display area 16d. Gathering information on points contained in this rectangular area allows the obtaining of a list of coordinate information where that tissue is present, thereby enabling extraction of an arbitrary tissue (FIG. 2, Step 6).

Tissue distributions in histogram space frequently overlap. A specified tissue can be efficiently extracted by selecting an area of a complex shape instead of a simple rectangular area. The creation of a library of tissue extraction patterns in this manner for each imaging site is clinically important.

(6) Clinical Application of Tissue Extraction

A common mask of a specific tissue can be obtained with each sequence using a list of coordinate information. Clinically important applications that are made possible by using this tissue mask are indicated below.

(6-1) Highlighting of Pathologic Tissue and Automatic Diagnosis

Since tumor tissue and the like does not exhibit a normal signal pattern, it appears in the histogram space as an island that is normally not observed. Therefore, the processing unit 14 is provided with a function for highlighting pathologic tissue exhibiting an abnormal signal pattern present among tissue extracted by the extraction unit 13. A specific example of a method thereof consists of standardizing signals from an island pattern in the histogram space, followed by displaying signals corresponding to an island having an abnormal signal pattern (island normally not observed) in a color such as red. As a result, the pathologic tissue is highlighted as a red mask region on the ordinary black and white image. This is easily understood by providing a function for switching highlighting on and off with software.

In actuality, a highlighting function is useful as a diagnostic aid for diagnosing not only on the basis of signal values, but also based on location, spread and the like. Since diagnosis can naturally be made from signal distribution alone if there is a clearly abnormal signal distribution, an automatic diagnostic function may also be provided that automatically presents the result of diagnosis to a user. However, a highlighting function that informs a user of clearly abnormal sites is thought to enable clinical use with greater confidence.

(6-2) Quantification of Target Tissue by Volume Measurement

Volume changes in the periosteum during rheumatoid arthritis or volume changes of a tumor are used to assess the effects of drugs and the like. Volume is normally calculated artificially by measuring length and width in a cross-section where the object appears the largest. This type of measurement method was adopted since the human eye is used to extract a specific tissue. In recent years, this type of measurement work has increased due to the vast amount of imaging testing being performed, thus increasing the burden on physicians having to interpret these images.

If it were possible to automatically extract a specific tissue area, the volume of even complex shapes could be measured automatically simply by counting the number of extracted voxels. Therefore, the processing unit 14 is provided with a function for measuring the volume of tissue extracted by the extraction unit 13 from the number of voxels belonging to the area of that tissue. By extracting the same site of histogram space for data sets of MRI tests performed before and after treatment of the same person and comparing tissue volume, it becomes possible to compare pathologic tissue with high reproducibility, thereby making this useful for assessing therapeutic effects.

(6-3) Creation of Actual Model and Navigation

A bone model is created by creating a full-size model from CT and MRI three-dimensional data, and pre-operative bending of the plate is carried out on that bone model. In addition, if the three-dimensional shape of the bone is known, it can also be used as data for navigation surgery. Although MRI three-dimensional data is inferior to CT three-dimensional data, if tissue is extracted using multiple sequences, extraction accuracy improves due to improved tissue contrast. Therefore, the processing unit 14 is provided with a function for generating three-dimensional data of tissue by extracting coordinate information of tissue extracted by the extraction unit 13. Although mask data, in which the presence of tissue is defined as 1 for the presence of tissue or 0 for the absence of tissue, is required in this case, according to the present invention, this can be realized simply by selecting an area from the histogram space and extracting coordinate information.

(6-4) Multi-sequence Color Mapping

If a plurality of sequences are respectively converted to RGB and displayed on the display unit 16, the plurality of sequences can be diagnosed simultaneously. Therefore, the processing unit 14 is provided with a function for generating a color map by correlating each cone cell with each MRI sequence, and respectively converting a plurality of the MRI sequences to RGB based on those correlations.

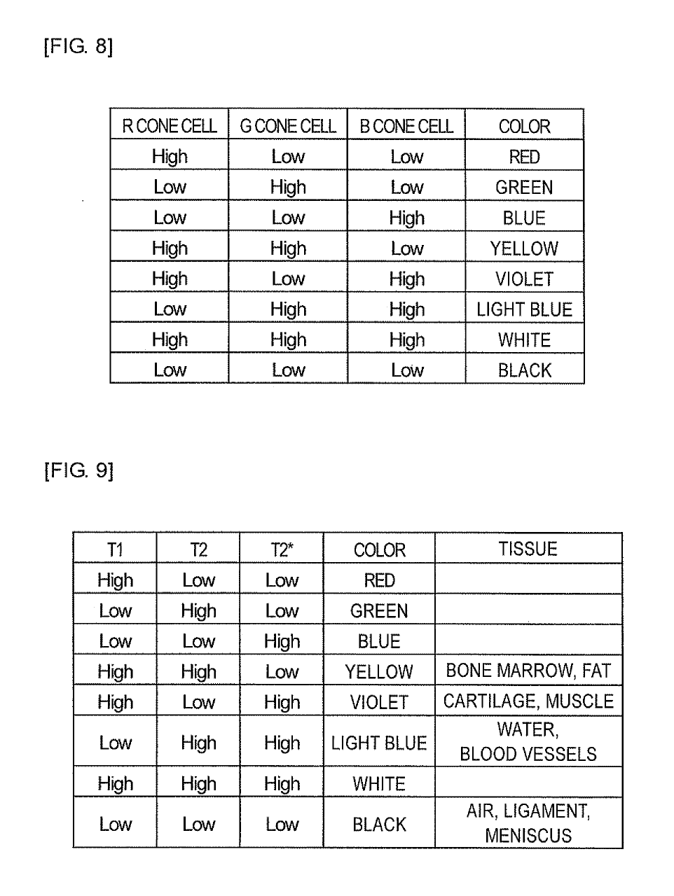

Namely, human photoreceptor cells consist of rod cells that perceive light and dark and cone cells that perceive color, and there are three types of cone cells consisting of “red”, “green” and “blue” that comprise the so-called primary colors. More specifically, as shown in FIG. 8, “color” is perceived by superposition of red, green and blue. Therefore, as shown in FIG. 9, R cone cells are correlated with T1 weighted images, G cone cells are correlated with T2 weighted images, and B cone cells are correlated, with T2* weighted images. These correlations can be realized by coloring T1 weighted images R, T2 weighted images G and T2* weighted images B in the display area 16b of FIG. 7. If each sequence is synthesized while in this state, since fat is displayed with yellow color and water is displayed with light blue color, each tissue can be classified with a natural color scheme.

(6-4-1) Since MRI signals are relative values, confusion results due to color tone changing each time unless each sequence is standardized. At this time, if signals are corrected and standardized based on tissue classification using island distribution in histogram space, results can be displayed in the same color tones for each test. MRI images are normally black and white images, and a physician interprets the images by changing the contrast. By providing a contrast correction function for each of the R, G and B signals, fine adjustments can be added when corrections in the histogram space do not proceed as desired.

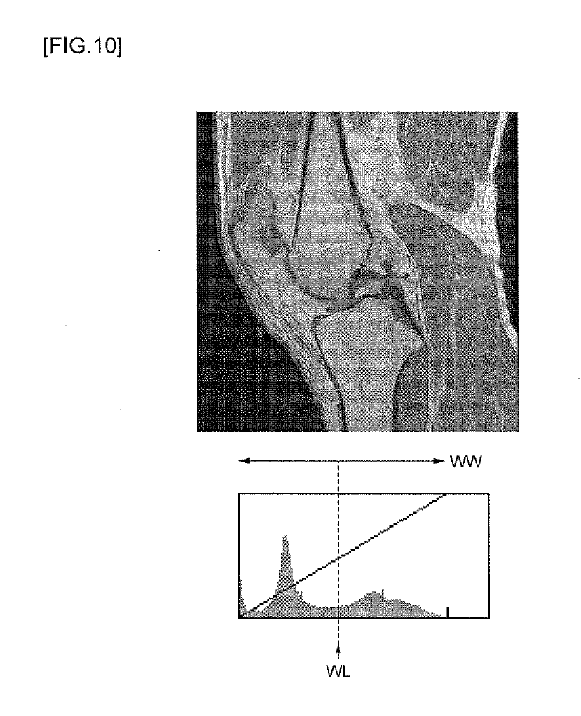

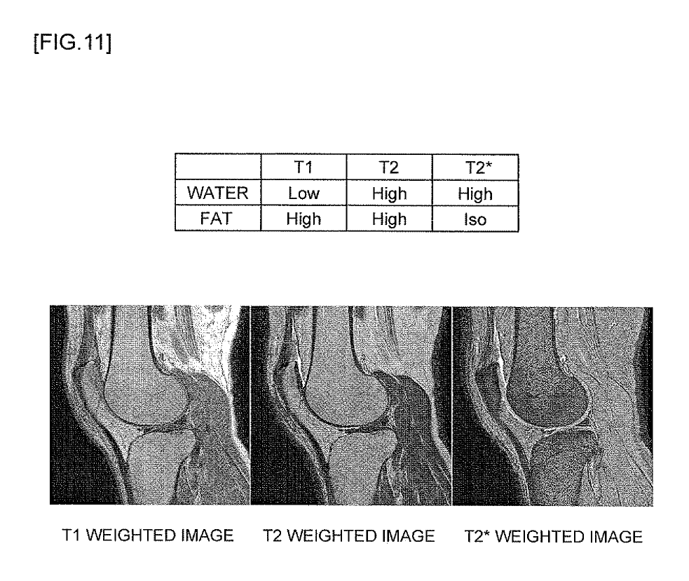

Although various methods can be considered for making these fine adjustments, since air signals are gathered at the minimum value of MRI signal values (signal value: 0), the manner in which the maximum value is set is important. Therefore, as shown in FIG. 10, two variables are set consisting of window level (WL) and window width (WW). The values of these two variables can be finely adjusted in the display area 16c of FIG. 7. In cases in which highly precise fine adjustment is required, gamma correction is set, and depending on the case, a tone curve is set with a freeform curve.

(6-4-2) Since human color vision employs a system by which R, G and B are respectively analyzed simultaneously with three types of cone cells, four or more colors cannot be analyzed simultaneously (although a small number of people are genetically capable of perceiving four colors, they are color blind for two primary colors). However, since MRI sequences frequently consist of four or more types, it is necessary to convert information of four dimensions or more to three-dimensional information in order to simultaneously display these MRI sequences.

The simplest method for accomplishing this consists of combining two specific sequences into one dimension. Two specific sequences can be combined by designating a conversion function in a two-dimensional signal space (Equation 1). Normally a simple linear transformation is used (Equation 2). There are cases in which conversion in space is effective for increasing display ability for targeting a specific tissue (Equation 3). Conversion that results in maximum color distance between islands in the histogram space is useful.

F(S1,S2)=mS (Equation 1)

F(S1,S2)=aS1+bS2 (Equation 2)

F(S1,S2,S3, . . . Sn)=(R,G,B) (Equation 3)

As has been described above, according to the tissue extraction system 10 in an embodiment of the present invention, tissue can be efficiently extracted since tissue isolation efficiency is improved by converting a plurality of MRI sequences to histogram space. Moreover, since the tissue extraction system 10 can be applied in various clinically important fields such as highlighting and automated diagnosis of pathologic tissue, quantification of target issue by measuring volume, creation of actual models and navigation, or multi-sequence color mapping, it can be said to be an invention having extremely high practical value. Since MRI allows imaging of various sequences, it also has the advantage of enabling numerous tests having different tissue contrast to be carried out.

Furthermore, although the case of using MRI images has been exemplified here, the present invention can also be applied to the case of using images other than MRI images, such as CT images, ultrasound images or electron microscope images, provided the configuration allows a plurality of signals to be obtained from the same coordinates.

Namely, although CT devices capture images using only a single energy source, dual energy imaging has appeared in recent years in which images are captured by arranging two sets of X-ray tubes and detectors respectively offset by 90°. As a result, two types of signals can be obtained for the same coordinates. It is predicted that the time will come when two or more types of signals will be able to be obtained as a result of future increases in the types of detectors. The present invention can be applied and effects similar to those previously described can be obtained even in the case of being able to obtain a plurality of signals from the same coordinates in this manner.

At present, CT three-dimensional display methods employ a method consisting of generating a three-dimensional display by setting transparency and color for specific signal values. For example, in the case of a three-dimensional display of bone, a library is created in advance such that bone signal values are assigned a transparency of 0 (namely, opaque) and appear white, other sites are assigned a transparency of 100, and intermediate sites are displayed by depicting gradations of color and transparency. As a result, three-dimensional images can be generated from CT data in a single step.

Three-dimensional images can be displayed automatically by expanding this method and providing a function for setting transparency and color at each point in histogram space. In the case of MRI, sequences can be converted to three-dimensional images having the same appearance each time by standardizing each sequence using island information. A function is required for specifying a standardization coefficient for each sequence since there are slight shifts even if island information is used. By selecting an area using a freeform curve in addition to a rectangle when specifying in histogram space, even close distances in the histogram space can be efficiently isolated. This system can also be created by expanding the area selection function of three-dimensional image processing software.

Furthermore, the present invention can not only be realized in the form of the tissue extraction system 10, but can also be realized in the form of a tissue extraction method that comprises a feature processing unit provided by this tissue extraction system 10 as a step thereof, or can be realized in the form of a tissue extraction program for executing these steps with a computer. Such a tissue extraction program can naturally be distributed on a CD-ROM or other recording medium or via a transfer medium such as the Internet.

### INDUSTRIAL APPLICABILITY

The present invention can be applied to a tissue extraction system required to efficiently extract tissue using images.

### Explanation of Numerals and Characters

10 Tissue extraction system

11 Imaging unit

12 Conversion unit

13 Extraction unit

14 Processing unit (highlighting unit, volume measurement unit, three-dimensional data generation unit, mapping unit)

15 Memory unit

16 Display unit

1. A tissue extraction system for extracting tissue of a subject using images, the system comprising:
an imaging unit for capturing a plurality of sequences by arranging coordinate axes;
a conversion unit for converting the plurality of sequences captured by the imaging unit into histogram space in which the sequences are described in terms of the coordinate axes; and
an extraction unit for extracting the tissue of the subject from the distribution of the histogram generated by the conversion unit.

2. The tissue extraction system according to claim 1, wherein the extraction unit calculates center points of islands in the histogram space, and carries out standardization based on the center points.

3. The tissue extraction system according to claim 1, wherein the extraction unit carries out labeling for correlating coordinates of the histogram space with the tissue.

4. The tissue extraction system according to claim 3, further comprising a highlighting unit for highlighting pathologic tissue exhibiting an abnormal signal pattern present in the tissue extracted by the extraction unit.

5. The tissue extraction system according to claim 3, further comprising a volume measurement unit for measuring a volume of the tissue extracted by the extraction unit from the number of voxels belonging to an area of the extracted tissue.

6. The tissue extraction system according to claim 3, further comprising a three-dimensional data generation unit for generating three-dimensional data by extracting coordinate information on the tissue extracted by the extraction unit.

7. The tissue extraction system according to claim 3, further comprising a map generation unit for generating a color map by correlating each cone cell with each sequence, and converting each of a plurality of the sequences to RGB based on those correlations.

8. The tissue extraction system according to claim 1, wherein the conversion unit integrates the plurality of sequences, arranges the data in a multi-sequence matrix, and develops the sequences by calculating frequency distributions in the histogram space.

9. A three-dimensional display method of a tissue extraction system for extracting tissue from a subject by using images, the method comprising:
converting a plurality of sequences captured by arranging coordinate axes into histogram space in which the sequences are described in terms of the coordinate axes, and designating a standardization coefficient for each sequence by selecting an area using a freeform curve in the case of standardizing each sequence using island information of the histogram space, and setting transparency and color at each point in the histogram space using a preliminarily created library, whereby resultantly in imaging space the same transparency and color are set to the same points in the histogram space and automatic conversion to a three-dimensional image is implemented by using the information.

10. The tissue extraction system according to claim 2, wherein the extraction unit carries out labeling for correlating coordinates of the histogram space with the tissue.

11. The tissue extraction system according to claim 10, further comprising a highlighting unit for highlighting pathologic tissue exhibiting an abnormal signal pattern present in the tissue extracted by the extraction unit.

12. The tissue extraction system according to claim 10, further comprising a volume measurement unit for measuring a volume of the tissue extracted by the extraction unit from the number of voxels belonging to an area of the extracted tissue.

13. The tissue extraction system according to claim 10, further comprising a three-dimensional data generation unit for generating three-dimensional data by extracting coordinate information on the tissue extracted by the extraction unit.

14. The tissue extraction system according to claim 10, further comprising a map generation unit for generating a color map by correlating each cone cell with each sequence, and converting each of a plurality of the sequences to RGB based on those correlations.
